SEQUENCE LISTING (11 sequences)

SEQ ID NO: 1 - DNA, 2949 bp, Artificial Sequence
  Genomic DNA of the gene OsCKX11
gagaggcagagcaagcgagcgagctgctgcacagtgacatcacggttacagagagagcttagctctgctcgggctcggctcagctcagctcagctgcagagagagagagacagagaaacaagaaacgcagcggcgagccaagatgatgctcgcgtacatggaccacgccgccgcggccgcggagccggacgccggcgccgagccggcggtggccgcggtcgacgcggccgagttcgcggcggcgatggacttcggcggcctggtgagcgcccgccccgccgccgtcgtccgcccggcgagctcggacgacgtggccagcgccatccgcgcggcggcgcgcaccgcgcacctgaccgtggccgcccgcggaaacggccactcggtggccgggcaggccatggcccgcggcggcctcgtcctcgacatgcgcgccctccctcgccgcatgcagctcgtcgtcgccccgtccggcgagaagttcgccgaagtcccgggcggcgcgctctgggaggaggtgctccactgggcagtgtcgaagcacggcctcgcccccgcctcctggacggactacctccgcctcaccgtcggcggcacgctctccaacggcggcgtgagcgggcaatccttccggtacgggccccaggtgtccaacgtcgcccagctcgaggtggtgaccggcgacggcgagtgccatgtctgctcccgctccgccgaccccgacctcttcttcgccgtcctcggcggcctcggccagttcggcgtcatcacccgcgcccgcatccctctctcccccgcgccccaaacggtaagcaccacaccaccacccaatcggaacgaacgacggcccaatcgcccggcggccgctgaccggcgagagctggctctgcaggtgcggtggacgcgggtggtgtacgcgagcttcgcggactacgcggcggacgcggagtggctggtgacgcggccgccgcacgaggcgttcgactacgtggagggattcgcgttcgtgcggagcgacgacccggtcaacggctggccaacggtgcccatcccggacggcgctcacttcgacgcctccctcctcccggccaacgccggcccggtgctctattgcctcgaggtcgccctgtaccaacgcggcggcggcggagacggcggtggcgacgacatggacaaggtacgtgagcgagtagtaattcccacgcgcggcggggggcattcccgtacatggtgtacttttctgggcggatgtctgcctccgtcgtgtatcccccccgctggattctgtgacgggtgcgtgctctgctcctcccgcgcgcgtgccgccaaaccacacacaccccctcccctgcccccacccacacccgccggtcgctgcctcgctcgcgcccaagccggatcacgcctcgatctcccgtgagccggggcgtgcgttgggcgttggcgtacaatgcggctcgcgctcgctgccgcgcccgtgacgacgcggatcccctgttttgtacacgcgcgggcgcacgctttgtcgcggtggtgacgcgggctgccgtttctctgtttcatttgggaggggggggtgtcgtctcgtgtcgtgctgatgatggcgtgtgtgtgtgtacgtgtggttggtttgcagagggtgggggagatgatgcggccgctcaagtacgtgcggggcctggagttcgcggcgggggtcgggtacgtggacttcctctcgcgcgtgaaccgggtggaggacgaggcccgccgcaacgggagctgggccgcgccgcacccgtggctcaacctcttcatctcctcacgcgacatcgccgccttcgaccgcgccgtcctcaacggcatgctcgccgacggcgtcgacgggcccatgctcatctaccccatgctcaagtccaagtgagtactactagtatactatttgtttatctcctgggatgggtttttgtttaatcggataattaattagcccatttggtccgtacttataatacgacgggggtttctggttgtcttccatcccgttctgttttggatttagccttgtcatatatctgccgccattaggatttagcagccactaaccccaggttgctatgattgatgtaaattcctttttcttcttttttttctctctctctctgtctcagtttgccgccaatgcacgcacgcacgcacacgagctgctaattaaaacgccccctaattaacacgtttgcgtgtgacaggtgggacccggccacgtcggtggcgctgccgaatggcgagatcttctacctggtggcgctgctccgattctgccggccctaccccggtggtggcccgccggtggacgagctggtggcgcagaacaacgcaatcattgacgcctgccggtccaacggctacgactacaagatatacttcccgagctaccacgcccagtccgactggtcgcgccacttcggcgccaagtggagccgcttcgtcgaccgcaaggcacgctacgacccgctcgccatcctcgcccccggccagaacatcttcgcccggaccccctcctccgtcgccgccgccgccgccgtgatcgtgtaagagacggatgatcgacgatggtgattatgctgtttgctgggttaattctggatgatggcgacgatgaggatgatggtgatgatggggatgaagaggagggatcgggacgagcacaatgatgatggtgatgatgatagggtcattgttaggtacatttgggaggggtgcaaaagagggaggtttcggttcgatgggatggacgacgtgtcaagggcagtagggccggcggctgtggctcggctctgcagcaggagttgcaaaagggaaaacgaaagatgtaaacgttttcctgctttgattctttttcttctcattccccctggtgagattgggacgcctttcgacggtgacacacatctcgtctcgttgttgggttaa

SEQ ID NO: 2 - DNA, 1557 bp, Artificial Sequence
  Encoding sequence of the gene OsCKX11
atgatgctcgcgtacatggaccacgccgccgcggccgcggagccggacgccggcgccgagccggcggtggccgcggtcgacgcggccgagttcgcggcggcgatggacttcggcggcctggtgagcgcccgccccgccgccgtcgtccgcccggcgagctcggacgacgtggccagcgccatccgcgcggcggcgcgcaccgcgcacctgaccgtggccgcccgcggaaacggccactcggtggccgggcaggccatggcccgcggcggcctcgtcctcgacatgcgcgccctccctcgccgcatgcagctcgtcgtcgccccgtccggcgagaagttcgccgacgtcccgggcggcgcgctctgggaggaggtgctccactgggcagtgtcgaagcacggcctcgcccccgcctcctggacggactacctccgcctcaccgtcggcggcacgctctccaacggcggcgtgagcgggcaatccttccggtacgggccccaggtgtccaacgtcgcccagctcgaggtggtgaccggcgacggcgagtgccatgtctgctcccgctccgccgaccccgacctcttcttcgccgtcctcggcggcctcggccagttcggcgtcatcacccgcgcccgcatccctctctcccccgcgccccaaacggtgcggtggacgcgggtggtgtacgcgagcttcgcggactacgcggcggacgcggagtggctggtgacgcggccgccgcacgaggcgttcgactacgtggagggattcgcgttcgtgcggagcgacgacccggtcaacggctggccaacggtgcccatcccggacggcgctcacttcgacgcctccctcctcccggccaacgccggcccggtgctctactgcctcgaggtcgccctgtaccaacgcggcggcggcggagacggcggtggcgacgacatggacaagagggtgggggagatgatgcggcagctcaagtacgtgcggggcctggagttcgcggcgggggtcgggtacgtggacttcctctcgcgcgtgaaccgggtggaggacgaggcccgccgcaacgggagctgggccgcgccgcacccgtggctcaacctcttcatctcctcacgcgacatcgccgccttcgaccgcgccgtcctcaacggcatgctcgccgacggcgtcgacgggcccatgctcatctaccccatgctcaagtccaagtgggacccggccacgtcggtggcgctgccgaatggcgagatcttctacctggtggcgctgctccgattctgccggccctaccccggtggtggcccgccggtggacgagctggtggcgcagaacaacgcaatcattgacgcctgccggtccaacggctacgactacaagatatacttcccgagctaccacgcccagtccgactggtcgcgccacttcggcgccaagtggagccgcttcgtcgaccgcaaggcacgctacgacccgctcgccatcctcgcccccggccagaacatcttcgcccggaccccctcctccgtcgccgccgccgccgccgtgatcgtgtaa

SEQ ID NO: 3 - protein, 518 aa, Artificial Sequence
  Amino acid sequence of the protein OsCKX11
MMLAYMDHAAAAAEPDAGAEPAVAAVDAAEFAAAMDFGGLVSARPAAVVRPASSDDVASAIRAAARTAHLTVAARGNGHSVAGQAMARGGLVLDMRALPRRMQLVVAPSGEKFADVPGGALWEEVLHWAVSKHGLAPASWTDYLRLTVGGTLSNGGVSGQSFRYGPQVSNVAQLEVVTGDGECHVCSRSADPDLFFAVLGGLGQFGVITRARIPLSPAPQTVRWTRVVYASFADYAADAEWLVTRPPHEAFDYVEGFAFVRSDDPVNGWPTVPIPDGAHFDASLLPANAGPVLYCLEVALYQRGGGGDGGGDDMDKRVGEMMRQLKYVRGLEFAAGVGYVDFLSRVNRVEDEARRNGSWAAPHPWLNLFISSRDIAAFDRAVLNGMLADGVDGPMLIYPMLKSKWDPATSVALPNGEIFYLVALLRFCRPYPGGGPPVDELVAQNNAIIDACRSNGYDYKIYFPSYHAQSDWSRHFGAKWSRFVDRKARYDPLAILAPGQNIFARTPSSVAAAAAVIV

SEQ ID NO: 4 - DNA, 24 bp, Artificial Sequence
  Forward primer sequence for the complementary primer sequence
ggcaaagttcgccgacgtcccggg

SEQ ID NO: 5 - DNA, 24 bp, Artificial Sequence
  Reverse primer sequence for the complementary primer sequence
aaaccccgggacgtcggcgaactt

SEQ ID NO: 6 - DNA, 20 bp, Artificial Sequence
  Forward primer sequence for PCR verification of final vector
attaaccctcactaaaggga

SEQ ID NO: 7 - DNA, 24 bp, Artificial Sequence
  Reverse primer sequence for PCR verification of final vector
aaaccccgggacgtcggcgaactt

SEQ ID NO: 8 - DNA, 20 bp, Artificial Sequence
  Forward primer sequence for PCR verification of final vector
acactttatgcttccggctc

SEQ ID NO: 9 - DNA, 24 bp, Artificial Sequence
  Reverse primer sequence for PCR verification of final vector
aaaccccgggacgtcggcgaactt

SEQ ID NO: 10 - DNA, 20 bp, Artificial Sequence
  Forward primer for PCR amplification
atggctgttttggaggtccg

SEQ ID NO: 11 - DNA, 20 bp, Artificial Sequence
  Reverse primer for PCR amplification
agcagacatggcactcgccg

(19) United States
(12) Patent Application Publication
(10) Pub. No.: US 2021/0324397 A1
(43) Pub. Date: Oct. 21, 2021

(54) A GENE OSCKX11 FOR CONTROLLING RICE GRAIN NUMBER AND USE THEREOF

(71) Applicant: Zhejiang Normal University, Jinhua (CN)

(72) Inventors: Kewei ZHANG, Jinhua (CN); Kaixuan PENG, Jinhua (CN); Wei ZHANG, Jinhua (CN); Fubin CUI, Jinhua (CN); Jiangzhe ZHAO, Jinhua (CN)

(73) Assignee: Zhejiang Normal University, Jinhua (CN)

(21) Appl. No.: 17/229,530

(22) Filed: Apr. 13, 2021

(30) Foreign Application Priority Data
Apr. 15, 2020 (CN) 202010296514.1

(51) Int. Cl. C12N 15/82; C12N 9/06

(52) U.S. Cl. CPC C12N 15/8261; C12N 15/8216; C12N 15/8213; C12N 9/0026; C12Y 105/99012

(57) ABSTRACT

The present disclosure belongs to the technical field of plant genetic engineering, and discloses a gene for controlling the rice grain number per panicle and its use. Nucleotide sequence of OsCKX11 is SEQ ID NO. 1, nucleotide sequence for coding protein region is SEQ ID NO. 2, amino acid sequence of the encoded protein is SEQ ID NO. 3. The disclosure constructs an OsCKX11-knocked-out vector using CRISPR/Cas9, and identifies multiple independent homozygous lines through PCR amplification and sequencing methods, and provides a mutant in which specific knockout of gene OsCKX11 of the rice leads to an increase in cytokinin levels and an increase in grain number per panicle. Based on the biological function of OsCKX11 to increase the rice grain number per panicle, methods such as gene editing, natural allele replacement, RNA interference, or molecular assisted breeding can be used to improve existing rice varieties.

### CROSS REFERENCE TO RELATED APPLICATION

This disclosure claims the priority of Chinese Patent Application NO. 202010296514.1 entitled A Gene OsCKX11 for controlling rice grain number and use thereof filed with the China National Intellectual Property Administration on Apr. 15, 2020, which is incorporated herein by reference in its entirety.

### TECHNICAL FIELD

The present disclosure belongs to the technical field of plant genetic engineering, and particularly relates to a gene for controlling the rice (Orazy sativa L) grain number and its use.

### BACKGROUND

As global population increases, food crisis facing mankind has become increasingly severe. Rice is one of the three staple crops in the world. Nearly half of the population uses rice as the main food in the world. Yield has always been an important economical trait in rice production and breeding. Rice yield is mainly determined by tillers, grain number per panicle and grain weight, among which grain number per panicle are a key factor in rice yield. Therefore, research on genes related to rice grain number can provide an important theoretical basis for increasing food production to ensure national food security.

Cytokinins are a class of small molecule plant hormones composed of N⁶-adenine derivatives and play an important role in plant growth and development, senescence, disease resistance, stress resistance and other life activities. Cytokinin oxidase is the main pathway to degrade cytokinin in plants. Loss or gain of the gene function of this enzyme will result in changes in the level of cytokinin in plants, which will affect normal growth and development of the plants. After the two genes of AtCKX3 and AtCKX5 in Arabidopsis thaliana are mutated simultaneously, the increase in cytokinin levels leads to an increase in the number of floral organs and enlargement of cells. The number of inflorescences in ckx3 and ckx5 double mutants increases significantly, and the number of fruit pods and inflorescences increased by nearly 60% when compared with that of the wild type. Therefore, the research on the function of cytokinin oxidase is essential for increasing the crop yield.

The cytokinin oxidase family of rice has 11 family members which are sequentially named OsCKX1-11. The functions of some members have been reported. The down-regulation of the OsCKX2 gene expression resulted in the increase of rice tillers, grain number per panicle, and grain weight, which significantly increased rice yield. Overexpression of the OsCKX4 gene resulted in a decrease in the mutant cytokinin level, an increase of root length and an increase in the number of crown roots. The expression of OsCKX9 gene can be induced by strigolactone and can regulate the level of cytokinin. The mutant with lost function in this gene presents a phenotype of increased tillers, smaller plant height, and reduced panicles. The functions of other rice cytokinin oxidases have not been elucidated.

At present, many transcription factors have been confirmed to be involved in the regulation of rice grain number per panicle. Rice LAX2 encodes the rice transcription factors, and its function is similar to LAX1 gene. In the lax2 mutant, the development of axillary meristem is affected, showing a sparse panicle phenotype with reduced grains per panicle, and the simultaneous mutation of LAX1 and LAX2 promotes the reduction of panicle branches, which indicates that there may be different ways to regulate the formation of panicle branches. GL6 encodes an AT-rich transcription factor in plants. This transcription factor regulates rice grain length and number of spikelets by promoting the proliferation of cells in young panicles and young grains. Overexpression of GL6 leads to the reduction of large grains and the grain number per panicle. It has been proved that GL6 interacts with subunit C53 of RNA polymerase III and transcription factor C1 to regulate the expression of genes related to development of rice grains.

In addition, some genes have been reported to regulate the grain number per panicle in rice. Rice GAD1 encodes a secreted polypeptide. Disruption of the conservative cysteine residues will cause loss of polypeptide function, and result in increased grains per panicle, short grains and no awns in cultivated rice. Similarly, mutations of the rice DEP1 gene encoding phosphatidylethanolamine binding protein can promote cell division and increase the grain number per panicle, resulting in an increased rice yield by 15%-20%. The rice GNP1 gene is a key gene for gibberellin synthesis. Variation in the GNP1 promoter region leads to an increase in the transcriptional activity of this gene, which increases the activity of cytokinin through feedback regulation, thereby increasing the grain number and yield of rice. The GNS4 gene encodes a cytochrome P450 protein. A single nucleotide deletion in the promoter region of this gene reduces the expression level of GNS4, resulting in a decrease in grain number and grain size.

In summary, both in monocot and dicot plants, cytokinin oxidase can regulate the level of cytokinin and affect rice panicles and grains. However, there are still some technical problems in this field. First, in addition to the reported mutants of osckx2, osckx4, and osckx9, there have been no related reports about osckx11 mutant. Second, among the 11 members of the rice cytokinin oxidase family, only OsCKX2 has been reported to be associated with regulation of grain number per panicle. The functions of the other 10 members are either unresolved or unrelated to regulation of grain number. Most of the OsCKX functions have not been reported. Therefore, it is urgent to solve the above problems.

### Difficulty in Solving the Above Technical Problems

To solve the above technical problems, firstly, it is necessary to create a rice mutant osckx11. Using the known rice Nipponbare genome sequence, specific sequences on the gene exons are selected to design knockout targets. CRISPR-Cas9 gene editing technology is used to obtain rice osckx11 mutants, and the homozygous mutants are identified. The obtained homozygous T2 generation mutants are planted in the field, the cytokinin content is determined, and the agronomic traits associated with rice grain number are counted.

### Significance of Solving the Above Technical Problems

The genetically modified rice material is obtained through gene editing technology. The stable genetic osckx11 homozygous mutant lines are obtained after 2-3 generations of self-pollination, which filled the gap in related materials. Research on the function of this gene will not only help reveal the biological functions of the rice cytokinin oxidase family, but also lay a scientific theoretical foundation for the improvement of high-yield and high-quality rice varieties.

### SUMMARY OF THE DISCLOSURE

In view of the problems in the prior art, the present disclosure provides a gene for controlling rice grain number per panicle and its use. In the present disclosure, the rice cytokinin oxidase gene OsCKX11 is knocked out specifically by using CRISPR/Cas9 technology, and a CKX gene for regulating the rice grain number per panicle, which is different from gene OsCKX2, is elucidated, providing a new way for genetic modification of rice.

It is realized in this way in the present disclosure.

In one aspect, the present disclosure provides a gene of OsCKX11 for controlling rice grain number per panicle, wherein the nucleotide sequence of the gene is SEQ ID NO.:1.

Further, the gene for controlling rice grain number per panicle further includes a DNA sequence that has 90% or more homology with the sequence SEQ ID NO.: 1.

Further, the gene for controlling rice grain number per panicle further comprises an allele or a gene derivative with one or more bases being altered produced by base substitution, deletion, or addition.

Further, the gene for controlling rice grain number per panicle further comprises: a DNA molecule capable of hybridizing with the DNA sequence of SEQ ID NO.: 1.

In another aspect, the present disclosure provides the use of the protein encoded by the gene for controlling rice grain number per panicle, and the encoded protein has the nucleotide sequence SEQ ID NO.: 2.

Further, the amino acid sequence of the encoded protein is SEQ ID NO.: 3.

The encoded protein further includes an amino acid sequence that has 90% or more of homology with the amino acid sequence of SEQ ID NO.: 3.

The encoded protein further includes proteins and protein analogs with one or more amino acid being altered, produced by amino acid substitution, deletion, and addition based on the amino acid sequence SEQ ID NO.: 3.

The encoded protein further includes a fusion protein formed by ligating the protein of SEQ ID NO.: 3 to other tag proteins.

In yet another aspect, the present disclosure provides a plant genetic transformation vector constructed by using the gene for controlling rice grain number, wherein the plant genetic transformation vector comprises an expression vector for up-regulating OsCKX11 and the expression vector for up-regulating OsCKX11 comprises a recombinant promoter or an expression vector for construction and fusion of organ-specific promoter;

the plant genetic transformation vector further comprising: a DNA sequence consisting of the sequence of SEQ ID NO.: 1, or a DNA sequence having 90% or more homology with the sequence of SEQ ID NO.: 1, or an allele or a gene derivative with one or more bases being altered produced by base substitution, deletion, or addition, or a DNA molecule capable of hybridizing with the DNA sequence of SEQ ID NO.: 1.

Further, the plant genetic transformation vectors further includes a vector for down-regulating OsCKX11 through CRISPR/Cas9 technology, T-DNA insertion technology, EMS mutagenesis, RNA interference technology, or gene silencing technology;

and the plant genetic transformation vector up-regulates or down-regulates the expression level or activity of the protein of SEQ ID NO.: 3 through a relevant protein regulator.

The plant genetic transformation vector up-regulates or down-regulates the expression level or activity of the protein shown in SEQ ID NO.: 3 through a relevant protein regulator.

In yet another aspect, the present disclosure provides a recombinant bacterium, a plant callus and a cell line, all of which are expressed by the plant genetic transformation vector.

In summary, the present disclosure has the following advantages and positive effects: by providing the gene OsCKX11 for controlling rice grain number per panicle and its use, the OsCKX11 that is capable of regulating rice grain number per panicle is described (FIG. 4 to FIG. 8). In the present disclosure, the OsCKX11 gene is specifically knocked out, and a dense-panicle rice line in a genetic background of Nipponbare (FIG. 1 and FIG. 4) is obtained. The present disclosure provides a genetic breeding method for reducing the expression of OsCKX11 or completely deleting the function of OsCKX11 to increase rice grains.

The present disclosure constructs a vector with OsCKX11 being knocked out with CRISPR/Cas9, and identifies multiple independent homozygous lines through PCR amplification and sequencing methods, and provides a mutant in which specific knockout of the rice OsCKX11 gene leads to an increase in cytokinin levels and an increase in grain number per panicle. Based on the biological function of OsCKX11 to increase the rice grain number per panicle, methods such as gene editing, natural allele replacement, RNA interference, T-DNA insertion, genetic transformation or molecular assisted breeding can be used to improve commercial rice varieties and increase the grain number per panicle, providing a theoretical foundation for breeding of high-yield rice varieties.

### BRIEF DESCRIPTION OF THE DRAWINGS

FIG. 1A shows the design position of the OsCKX11 specific target.

FIG. 1B is the identification of the mutation mode of the osckx11 mutant, wherein the rectangle section indicates the region with mutated amino acids.

FIG. 2 is a schematic diagram of the construction of vector in which OsCKX11 gene is specifically knocked out provided by an embodiment of the present disclosure.

FIG. 2 shows the electrophoresis profile for PCR verification of ligation of final vector to OsCKX11 target fragment. Lane M represents DL5000 DNA Marker, 1-11 represents different single colonies, 12 represents positive control, and 13 represents negative control.

FIG. 3 is a schematic diagram of the results of quantification of cytokinin content in young leaves of homozygous mutants of osckx11 provided by an embodiment of the present disclosure.

In FIG. 3, tZ is trans-zeatin, cZ is cis-zeatin, cZR is cis-zeatin ribose, tZR is trans-zeatin ribose, iP is isopentenyl adenine, iPR is isopentenyl adenine ribose, DHZ is dihydrozeatin. SD (n=3), *P≤0.05, **P≤0.01, T test, FW represents fresh weight.

FIG. 4A shows (from left to right) the Nipponbare wild type and three independent osckx11 mutant lines.

FIG. 4B shows the panicles on the rice plant, wherein the left part shows the panicles on the Nipponbare wild type plant, and the right part shows the panicles on the osckx11 mutant plant.

FIG. 5 is a graph showing the statistics of the grain number per panicle of the osckx11 homozygous mutant provided by the embodiment of the present disclosure. The figure shows the number of seeds per panicle. WT represents Nipponbare wild type, and osckx11-1, 2 and 3 are three independent osckx11 mutant lines. SD (n=15), *P≤0.05, **P≤0.01, T test.

FIG. 6 is a graph showing the statistics of the grain number per plant of the osckx11 homozygous mutant of provided by the embodiment of the present disclosure. The figure shows the grain number on a single plant. WT represents Nipponbare wild type, and osckx11-1, 2 and 3 are three independent osckx11 mutant lines. SD (n=15), *P≤0.05, **P≤0.01, T test.

FIG. 7 is a graph showing the statistics of the number of primary branches of osckx11 homozygous mutants provided by an embodiment of the present disclosure. The figure shows the statistics of the number of primary branches on a single panicle. WT represents Nipponbare wild type, and osckx11-1, 2 and 3 are three independent osckx11 mutant lines. SD (n=15), *P≤0.05, **P≤0.01, T test.

FIG. 8 is a graph showing the yield per plant of osckx11 homozygous mutants provided by the embodiment of the present disclosure. The figure shows the weight statistics of the grain weight per line. WT represents Nipponbare wild type, and osckx11-1, 2 and 3 are three independent osckx11 mutant lines. SD (n=15), *P≤0.05, **P≤0.01, T test.

### DETAILED DESCRIPTION OF THE EMBODIMENTS

In order to bring out the objectives, technical solutions and advantages of the present disclosure more clearly, the present disclosure will be further described in detail below in conjunction with embodiments. It should be understood that the specific embodiments described herein are only used to explain the present disclosure, but not to limit the present disclosure.

In view of the problems in the prior art, the present disclosure provides a gene OsCKX11 for controlling rice grain number per panicle and its use. The present disclosure will be described in detail below with reference to the drawings.

The nucleotide sequence of the gene for controlling grain number per panicle provided by the embodiment of the present disclosure is SEQ ID NO.: 1.

The gene sequence of SEQ ID NO.: 1 is set forth below:

| | |
|---|---|
| 1 | GAGAGGCAGA GCAAGCGAGC GAGCTGCTGC ACAGTGACAT CACGGTTACA GAGAGAGCTT |
| 61 | AGCTCTGCTC GGGCTCGGCT CAGCTCAGCT CAGCTGCAGA GAGAGAGAGA CAGAGAAACA |
| 121 | AGAAACGCAG CGGCGAGCCA AGATGATGCT CGCGTACATG GACCACGCCG CCGCGGCCGC |
| 181 | GGAGCCGGAC GCCGGCGCCG AGCCGGCGGT GGCCGCGGTC GACGCGGCCG AGTTCGCGGC |
| 241 | GGCGATGGAC TTCGGCGGCC TGGTGAGCGC CCGCCCCGCC GCCGTCGTCC GCCCGGCGAG |
| 301 | CTCGGACGAC GTGGCCAGCG CCATCCGCGC GGCGGCGCGC ACCGCGCACC TGACCGTGGC |
| 361 | CGCCCGCGGA AACGGCCACT CGGTGGCCGG GCAGGCCATG GCCCGCGGCG GCCTCGTCCT |
| 421 | CGACATGCGC GCCCTCCCTC GCCGCATGCA GCTCGTCGTC GCCCCGTCCG GCGAGAAGTT |
| 481 | CGCCGAAGTC CCGGGCGGCG CGCTCTGGGA GGAGGTGCTC CACTGGGCAG TGTCGAAGCA |
| 541 | CGGCCTCGCC CCCGCCTCCT GGACGGACTA CCTCCGCCTC ACCGTCGGCG GCACGCTCTC |
| 601 | CAACGGCGGC GTGAGCGGGC AATCCTTCCG GTACGGGCCC CAGGTGTCCA ACGTCGCCCA |
| 661 | GCTCGAGGTG GTGACCGGCG ACGGCGAGTG CCATGTCTGC TCCCGCTCCG CCGACCCCGA |
| 721 | CCTCTTCTTC GCCGTCCTCG GCGGCCTCGG CCAGTTCGGC GTCATCACCC GCGCCCGCAT |
| 781 | CCCTCTCTCC CCCGCGCCCC AAACGGTAAG CACCACACCA CCACCCAATC GGAACGAACG |
| 841 | ACGGCCCAAT CGCCCGGCGG CCGCTGACCG GCGAGAGCTG GCTCTGCAGG TGCGGTGGAC |
| 901 | GCGGGTGGTG TACGCGAGCT TCGCGGACTA CGCGGCGGAC GCGGAGTGGC TGGTGACGCG |
| 961 | GCCGCCGCAC GAGGCGTTCG ACTACGTGGA GGGATTCGCG TTCGTGCGGA GCGACGACCC |
| 1021 | GGTCAACGGC TGGCCAACGG TGCCCATCCC GGACGGCGCT CACTTCGACG CCTCCCTCCT |
| 1081 | CCCGGCCAAC GCCGGCCCGG TGCTCTATTG CCTCGAGGTC GCCCTGTACC AACGCGGCGG |
| 1141 | CGGCGGAGAC GGCGGTGGCG ACGACATGGA CAAGGTACGT GAGCGAGTAG TAATTCCCAC |
| 1201 | GCGCGGCGGG GGGCATTCCC GTACATGGTG TACTTTTCTG GGCGGATGTC TGCCTCCGTC |
| 1261 | GTGTATCCCC CCCGCTGGAT TCTGTGACGG GTGCGTGCTC TGCTCCTCCC GCGCGCGTGC |
| 1321 | CGCCAAACCA CACACACCCC CTCCCCTGCC CCCACCCACA CCCGCCGGTC GCTGCCTCGC |
| 1381 | TCGCGCCCAA GCCGGATCAC GCCTCGATCT CCCGTGAGCC GGGGCGTGCG TTGGGCGTTG |
| 1441 | GCGTACAATG CGGCTCGCGC TCGCTGCCGC GCCCGTGACG ACGCGGATCC CCTGTTTTGT |
| 1501 | ACACGCGCGG GCGCACGCTT TGTCGCGGTG GTGACGCGGG CTGCCGTTTC TCTGTTTCAT |
| 1561 | TTGGGAGGGG GGGGTGTCGT CTCGTGTCGT GCTGATGATG GCGTGTGTGT GTGTACGTGT |
| 1621 | GGTTGGTTTG CAGAGGGTGG GGGAGATGAT GCGGCCGCTC AAGTACGTGC GGGGCCTGGA |
| 1681 | GTTCGCGGCG GGGGTCGGGT ACGTGGACTT CCTCTCGCGC GTGAACCGGG TGGAGGACGA |
| 1741 | GGCCCGCCGC AACGGGAGCT GGGCCGCGCC GCACCCGTGG CTCAACCTCT TCATCTCCTC |
| 1801 | ACGCGACATC GCCGCCTTCG ACCGCGCCGT CCTCAACGGC ATGCTCGCCG ACGGCGTCGA |
| 1861 | CGGGCCCATG CTCATCTACC CCATGCTCAA GTCCAAGTGA GTACTACTAG TATACTATTT |
| 1921 | GTTTATCTCC TGGGATGGGT TTTTGTTTAA TCGGATAATT AATTAGCCCA TTTGGTCCGT |
| 1981 | ACTTATAATA CGACGGGGGT TTCTGGTTGT CTTCCATCCC GTTCTGTTTT GGATTTAGCC |
| 2041 | TTGTCATATA TCTGCCGCCA TTAGGATTTA GCAGCCACTA ACCCCAGGTT GCTATGATTG |
| 2101 | ATGTAAATTC CTTTTTCTTC TTTTTTTTCT CTCTCTCTCT GTCTCAGTTT GCCGCCAATG |
| 2161 | CACGCACGCA CGCACACGAG CTGCTAATTA AAACGCCCCC TAATTAACAC GTTTGCGTGT |
| 2221 | GACAGGTGGG ACCCGGCCAC GTCGGTGGCG CTGCCGAATG GCGAGATCTT CTACCTGGTG |
| 2281 | GCGCTGCTCC GATTCTGCCG GCCCTACCCC GGTGGTGGCC CGCCGGTGGA CGAGCTGGTG |
| 2341 | GCGCAGAACA ACGCAATCAT TGACGCCTGC CGGTCCAACG GCTACGACTA CAAGATATAC |
| 2401 | TTCCCGAGCT ACCACGCCCA GTCCGACTGG TCGCGCCACT TCGGCGCCAA GTGGAGCCGC |
| 2461 | TTCGTCGACC GCAAGGCACG CTACGACCCG CTCGCCATCC TCGCCCCCGG CCAGAACATC |
| 2521 | TTCGCCCGGA CCCCCTCCTC CGTCGCCGCC GCCGCCGCCG TGATCGTGTA AGAGACGGAT |
| 2581 | GATCGACGAT GGTGATTATG CTGTTTGCTG GGTTAATTCT GGATGATGGC GACGATGAGG |
| 2641 | ATGATGGTGA TGATGGGGAT GAAGAGGAGG GATCGGGACG AGCACAATGA TGATGGTGAT |
| 2701 | GATGATAGGG TCATTGTTAG GTACATTTGG GAGGGGTGCA AAAGAGGGAG GTTTCGGTTC |
| 2761 | GATGGGATGG ACGACGTGTC AAGGGCAGTA GGGCCGGCGG CTGTGGCTCG GCTCTGCAGC |
| 2821 | AGGAGTTGCA AAAGGGAAAA CGAAAGATGT AAACGTTTTC CTGCTTTGAT TCTTTTTCTT |
| 2881 | CTCATTCCCC CTGGTGAGAT TGGGACGCCT TTCGACGGTG ACACACATCT CGTCTCGTTG |
| 2941 | TTGGGTTAA |

The gene for controlling rice grain number per panicle provided by the embodiment of the present disclosure also includes a DNA sequence that has 90% or more homology with the DNA sequence of SEQ ID NO.: 1.

The gene for controlling rice grain number per panicle provided by the embodiments of the present disclosure also includes one or more base-altered alleles or gene derivatives produced by base substitution, deletion, or addition.

The gene for controlling rice grain number per panicle provided by the embodiments of the present disclosure also includes DNA molecules that are capable of hybridizing with the DNA sequence of SEQ ID NO.: 1.

The nucleotide sequence of the protein encoded by using the gene for controlling rice grain number per panicle in the embodiment of the present disclosure is SEQ ID NO.: 2.

The gene sequence of SEQ ID NO.: 2 is set forth below:

| | |
|---|---|
| 1 | ATGATGCTCG CGTACATGGA CCACGCCGCC GCGGCCGCGG AGCCGGACGC CGGCGCCGAG |
| 61 | CCGGCGGTGG CCGCGGTCGA CGCGGCCGAG TTCGCGGCGG CGATGGACTT CGGCGGCCTG |
| 121 | GTGAGCGCCC GCCCCGCCGC CGTCGTCCGC CCGGCGAGCT CGGACGACGT GGCCAGCGCC |
| 181 | ATCCGCGCGG CGGCGCGCAC CGCGCACCTG ACCGTGGCCG CCCGCGGAAA CGGCCACTCG |
| 241 | GTGGCCGGGC AGGCCATGGC CCGCGGCGGC CTCGTCCTCG ACATGCGCGC CCTCCCTCGC |
| 301 | CGCATGCAGC TCGTCGTCGC CCCGTCCGGC GAGAAGTTCG CCGACGTCCC GGGCGGCGCG |
| 361 | CTCTGGGAGG AGGTGCTCCA CTGGGCAGTG TCGAAGCACG GCCTCGCCCC CGCCTCCTGG |
| 421 | ACGGACTACC TCCGCCTCAC CGTCGGCGGC ACGCTCTCCA ACGGCGGCGT GAGCGGGCAA |
| 481 | TCCTTCCGGT ACGGGCCCCA GGTGTCCAAC GTCGCCCAGC TCGAGGTGGT GACCGGCGAC |
| 541 | GGCGAGTGCC ATGTCTGCTC CCGCTCCGCC GACCCCGACC TCTTCTTCGC CGTCCTCGGC |
| 601 | GGCCTCGGCC AGTTCGGCGT CATCACCCGC GCCCGCATCC CTCTCTCCCC CGCGCCCCAA |
| 661 | ACGGTGCGGT GGACGCGGGT GGTGTACGCG AGCTTCGCGG ACTACGCGGC GGACGCGGAG |
| 721 | TGGCTGGTGA CGCGGCCGCC GCACGAGGCG TTCGACTACG TGGAGGGATT CGCGTTCGTG |
| 781 | CGGAGCGACG ACCCGGTCAA CGGCTGGCCA ACGGTGCCCA TCCCGGACGG CGCTCACTTC |
| 841 | GACGCCTCCC TCCTCCCGGC CAACGCCGGC CCGGTGCTCT ACTGCCTCGA GGTCGCCCTG |
| 901 | TACCAACGCG GCGGCGGCGG AGACGGCGGT GGCGACGACA TGGACAAGAG GGTGGGGGAG |
| 961 | ATGATGCGGC AGCTCAAGTA CGTGCGGGGC CTGGAGTTCG CGGCGGGGGT CGGGTACGTG |
| 1021 | GACTTCCTCT CGCGCGTGAA CCGGGTGGAG GACGAGGCCC GCCGCAACGG GAGCTGGGCC |
| 1081 | GCGCCGCACC CGTGGCTCAA CCTCTTCATC TCCTCACGCG ACATCGCCGC CTTCGACCGC |
| 1141 | GCCGTCCTCA ACGGCATGCT CGCCGACGGC GTCGACGGGC CCATGCTCAT CTACCCCATG |
| 1201 | CTCAAGTCCA AGTGGGACCC GGCCACGTCG GTGGCGCTGC CGAATGGCGA GATCTTCTAC |
| 1261 | CTGGTGGCGC TGCTCCGATT CTGCCGGCCC TACCCCGGTG GTGGCCCGCC GGTGGACGAG |
| 1321 | CTGGTGGCGC AGAACAACGC AATCATTGAC GCCTGCCGGT CCAACGGCTA CGACTACAAG |
| 1381 | ATATACTTCC CGAGCTACCA CGCCCAGTCC GACTGGTCGC GCCACTTCGG CGCCAAGTGG |
| 1441 | AGCCGCTTCG TCGACCGCAA GGCACGCTAC GACCCGCTCG CCATCCTCGC CCCCGGCCAG |
| 1501 | AACATCTTCG CCCGGACCCC CTCCTCCGTC GCCGCCGCCG CCGCCGTGAT CGTGTAA |

The regional sequence of encoded protein provided by the embodiment of the present disclosure also includes a DNA sequence that has 90% or more homology with the DNA sequence of SEQ ID NO.: 1.

The encoded protein region sequence provided by the embodiment of the present disclosure also includes one or more base-altered alleles or gene derivatives produced by base substitution, deletion, and addition.

The amino acid sequence of the protein encoded by using the gene for controlling rice grain number per panicle in the embodiment of the present disclosure is SEQ ID NO.:3.

The gene sequence of SEQ ID NO.:3 is set forth below:

| | |
|---|---|
| 1 | MET MET Leu Ala Tyr MET Asp His Ala Ala Ala Ala Ala Glu Pro Asp Ala Gly |
| 19 | Ala Glu Pro Ala Val Ala Ala Val Asp Ala Ala Glu Phe Ala Ala Ala MET Asp |
| 37 | Phe Gly Gly Leu Val Ser Ala Arg Pro Ala Ala Val Val Arg Pro Ala Ser Ser |
| 55 | Asp Asp Val Ala Ser Ala Ile Arg Ala Ala Ala Arg Thr Ala His Leu Thr Val |
| 73 | Ala Ala Arg Gly Asn Gly His Ser Val Ala Gly Gln Ala MET Ala Arg Gly Gly |
| 91 | Leu Val Leu Asp MET Arg Ala Leu Pro Arg Arg MET Gln Leu Val Val Ala Pro |
| 109 | Ser Gly Glu Lys Phe Ala Asp Val Pro Gly Gly Ala Leu Trp Glu Glu Val Leu |
| 127 | His Trp Ala Val Ser Lys His Gly Leu Ala Pro Ala Ser Trp Thr Asp Tyr Leu |
| 145 | Arg Leu Thr Val Gly Gly Thr Leu Ser Asn Gly Gly Val Ser Gly Gln Ser Phe |
| 163 | Arg Tyr Gly Pro Gln Val Ser Asn Val Ala Gln Leu Glu Val Val Thr Gly Asp |
| 181 | Gly Glu Cys His Val Cys Ser Arg Ser Ala Asp Pro Asp Leu Phe Phe Ala Val |
| 199 | Leu Gly Gly Leu Gly Gln Phe Gly Val Ile Thr Arg Ala Arg Ile Pro Leu Ser |
| 217 | Pro Ala Pro Gln Thr Val Arg Trp Thr Arg Val Val Tyr Ala Ser Phe Ala Asp |
| 235 | Tyr Ala Ala Asp Ala Glu Trp Leu Val Thr Arg Pro Pro His Glu Ala Phe Asp |
| 253 | Tyr Val Glu Gly Phe Ala Phe Val Arg Ser Asp Asp Pro Val Asn Gly Trp Pro |
| 271 | Thr Val Pro Ile Pro Asp Gly Ala His Phe Asp Ala Ser Leu Leu Pro Ala Asn |
| 289 | Ala Gly Pro Val Leu Tyr Cys Leu Glu Val Ala Leu Tyr Gln Arg Gly Gly Gly |
| 307 | Gly Asp Gly Gly Gly Asp Asp MET Asp Lys Arg Val Gly Glu MET MET Arg Gln |
| 325 | Leu Lys Tyr Val Arg Gly Leu Glu Phe Ala Ala Gly Val Gly Tyr Val Asp Phe |
| 343 | Leu Ser Arg Val Asn Arg Val Glu Asp Glu Ala Arg Arg Asn Gly Ser Trp Ala |
| 361 | Ala Pro His Pro Trp Leu Asn Leu Phe Ile Ser Ser Arg Asp Ile Ala Ala Phe |
| 379 | Asp Arg Ala Val Leu Asn Gly MET Leu Ala Asp Gly Val Asp Gly Pro MET Leu |
| 397 | Ile Tyr Pro MET Leu Lys Ser Lys Trp Asp Pro Ala Thr Ser Val Ala Leu Pro |
| 415 | Asn Gly Glu Ile Phe Tyr Leu Val Ala Leu Leu Arg Phe Cys Arg Pro Tyr Pro |
| 433 | Gly Gly Gly Pro Pro Val Asp Glu Leu Val Ala Gln Asn Asn Ala Ile Ile Asp |
| 451 | Ala Cys Arg Ser Asn Gly Tyr Asp Tyr Lys Ile Tyr Phe Pro Ser Tyr His Ala |
| 469 | Gln Ser Asp Trp Ser Arg His Phe Gly Ala Lys Trp Ser Arg Phe Val Asp Arg |
| 487 | Lys Ala Arg Tyr Asp Pro Leu Ala Ile Leu Ala Pro Gly Gln Asn Ile Phe Ala |
| 505 | Arg Thr Pro Ser Ser Val Ala Ala Ala Ala Ala Val Ile Val |

The encoded protein provided by the embodiment of the present disclosure also includes an amino acid sequence that has 90% or more homology with the amino acid sequence of SEQ ID NO.: 3.

The encoded protein provided by the embodiment of the present disclosure also includes proteins and protein analogs with one or more amino acid being altered produced by amino acid substitution, deletion, and addition based on the amino acid sequence of SEQ ID NO.: 3.

The encoded protein provided by the embodiment of the present disclosure also includes a fusion protein formed by ligating the protein of SEQ ID NO.: 3 to other tag proteins.

In the embodiments of the present disclosure, it is provided a plant genetic transformation vector constructed by using the gene OsCKX11 for controlling rice grain number per panicle, and the plant genetic transformation vector includes an OsCKX11 up-regulated expression vector, such as a recombinant promoter (for example, CaMV 35S promoter) or an expression vector for construction and fusion of organ-specific promoter.

The vector provided in the embodiment of the present disclosure comprises a DNA sequence consisting of the sequence of SEQ ID NO.: 1, or a DNA sequence that has 90% or more homology with the sequence of SEQ ID NO.: 1, or an allele or a gene derivative with one or more bases being altered produced by base substitution, deletion, or addition, or a DNA molecule capable of hybridizing with the DNA sequence of SEQ ID NO.: 1.

The plant genetic transformation vector provided by the embodiment of the present disclosure includes a down-regulated gene expression vector for down-regulating expression of the gene of SEQ ID NO.: 1 through CRISPR/Cas9 technology, T-DNA insertion technology, EMS mutagenesis, RNA interference technology, gene silencing technology.

The amount of expression or the activity of protein as shown in SEQ ID NO.: 3 is up-regulated or down-regulated by related protein regulators.

The present disclosure will be further described below in conjunction with examples.

In the present disclosure, a function-deficient osckx11 rice mutant is obtained by specifically adding or deleting one or more nucleotide bases in the coding region of rice OsCKX11 protein, and the rice grain number per panicle is significantly increased.

This is realized by the following steps.

Designing a gene knockout target in the OsCKX11 protein coding region, synthesizing a target sequence, ligating the target sequence to pC1300-Cas9 vector to construct an OsCKX11 specific knockout vector. The method is described in detail in Example 1.

Transforming the receptor of the transgenic rice Nipponbare with the successfully constructed OsCKX11 gene-specific knockout vector by Jiangsu Baige Gene Technology Co., Ltd., obtaining a transformed plant.

Extracting DNA of a transformed rice seedling, amplifying the fragments near the OsCKX11 gene target by means of PCR technology, sending to Hangzhou Qingke Biotechnology Company for full-length sequencing, and comparing the sequences to obtain a homozygous mutant. The method will be described in detail in Example 2.

Identifying the correct homozygous mutants and performing cytokinin quantification to obtain an independent genetic strain with reduced cytokinin. The method will be described in detail in Example 3.

Selecting three independent mutant lines for field breeding to obtain a T2 generation plant for field breeding again, counting and analyzing related phenotypes of grains on panicle. The method will be described in detail in Example 4 and Example 5.

The fusion expression vector constructed by using the OsCKX11 gene and other regulatory elements such as recombinant promoters or organ-specific promoters provided by the present disclosure, the method for regulating the rice grain number per panicle by using OsCKX11 provided by the present disclosure using a transgenic technology, antisense RNA, RNAi, T-DNA insertion and CRISPR/Cas9 technology, and the recombinant vector, the recombinant vector cell line and the recombinant bacteria carrying the OsCKX11 gene provided by the present disclosure, all fall within the protection scope of the present disclosure.

### Example 1

Design of OsCKX11 Knockout Target and Vector Construction

The gene OsCKX11 has an accession number of LOC_Os08g35860, and its gene function has not been elucidated yet. The deoxynucleotide sequence of the gene was queried through the Rice Genome Browser (http://rice.plantbiology.msu.edu), and the deoxynucleotide sequence of the gene is shown as SEQ ID NO.: 1 in the sequence listing, partial deoxynucleotide sequence of the protein encoded by the gene is shown as sequence SEQ ID NO.: 2 in the sequence listing, and the amino acid sequence of the protein encoded by the gene is shown as SEQ ID NO.: 3 in the sequence listing. The nucleotide sequence of gene OsCKX11 has 2949 bp, including four exons and three introns, as shown in SEQ ID NO.: 1 in the sequence listing.

Design of Specific Knockout Target

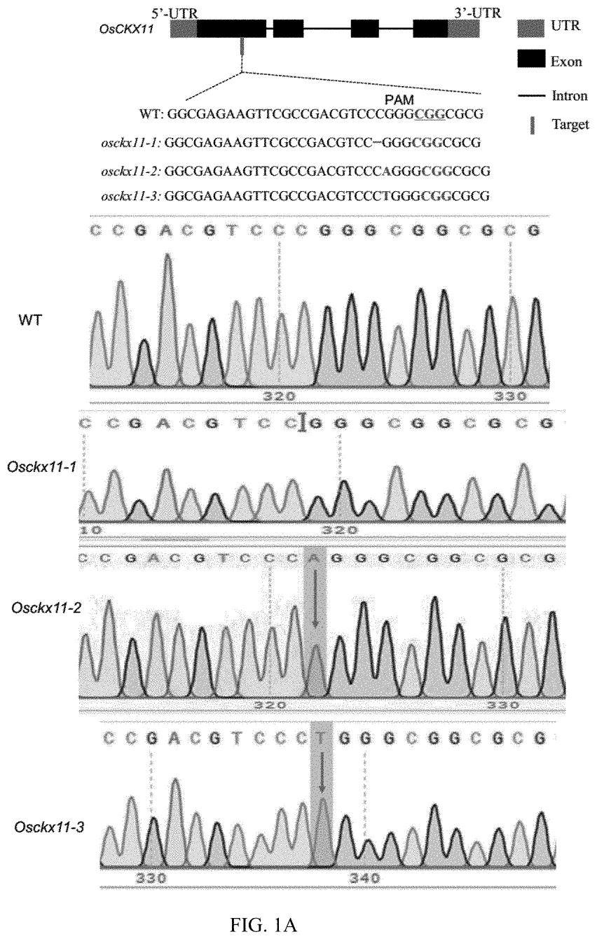

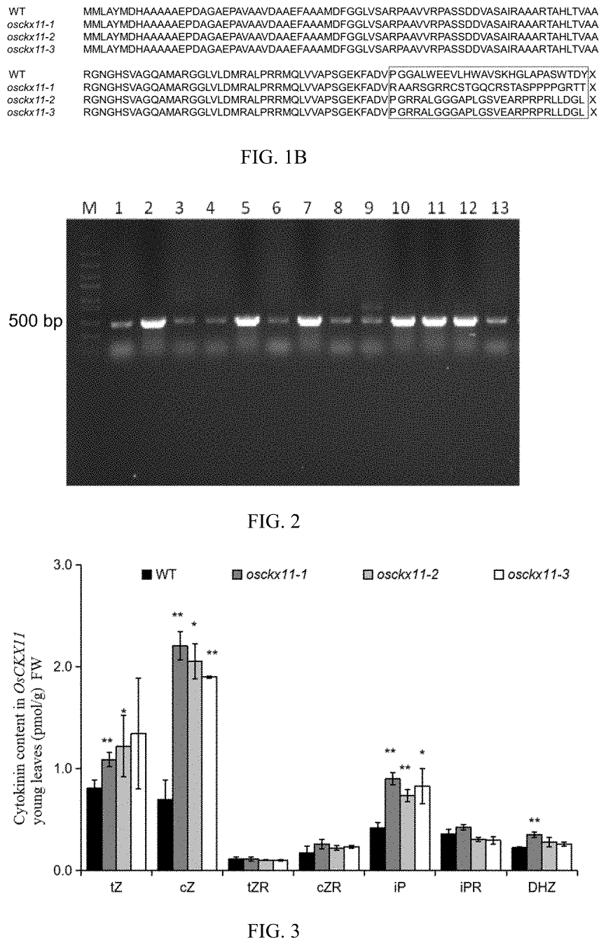

Log in to the CRISPR-PLANT website (https://www.genome.arizona.edu/crispr/CRISPRsearch.html) and design a specific knockout primers based on the deoxynucleotide sequence of the gene OsCKX11 as found. Design knockout target in the first exon, and in the target the forward and reverse primers are fully complementary, the PAM sequence of the forward primer sequence is CGG, the 5′ end bases of the forward primer is 333 bp away from the ATG initiation codon of the gene, as shown in FIG. 1A. The complementary primer sequences SEQ ID NO.: 4 and SEQ ID NO.: 5 for the target areas follows:

Forward primer of target:.
5'-GGCA AAGTTCGCCGACGTCCCGGG-3' (underlined is
the primer adapter to construct the intermediate
vector)
Reverse primer of target:.
5'-AAAC CCCGGGACGTCGGCGAACTT-3' (underlined is
the primer adapter to construct the intermediate
vector)
The gene sequences of SEQ ID NO.: 4 and SEQ ID
NO.: 5 are:
SEQ ID NO.: 4:
Forward primer:
GGCA AAGTTCGCCGACGTCCCGGG
SEQ ID NO.: 5:
Reverse primer of target:
AAAC CCCGGGACGTCGGCGAACTT

Construction of OsCKX11 Gene Knockout Vector

The CRISPR/Cas9 gene editing technology involved in the present disclosure may be referred to the rice multiple gene knockout system (Kejian Wang's Research Group of China Rice Research Institute). The intermediate vector SK-gRNA and the final vector pC1300-Cas9 were all obtained from Kejian Wang's research group of China National Rice Research Institute.

Ligating OsCKX11 Target to Intermediate Carrier SK-gRNA

AarI restriction endonuclease (purchased from Thermo Fisher Scientific, please refer to the product instructions for specific usage and dosage) was used to digest the SK-gRNA plasmid, the digestion system was as follows: 10× Buffer AarI 5 μL, 50× oligonucleotide 1 μL, AarI 1 μL, SK-gRNA 1-2 μL. The rest was made up to 50 μL system with ddH₂O and digestion was conducted at 37° C. for 3-6 h.

The forward and reverse primers of the target (concentration 100 μM) 20 μL each were mixed for denaturation and annealing, denatured at 100° C. for 5 min, and cooled to room temperature.

The cooled target primer was ligated to the SK-gRNA recovered by enzyme digestion with T4 DNA ligase (purchased from NEB company, specific usage and dosage may be referred to the product instruction manual).

The ligation product was transformed into Escherichia coli DH5α, spread on a 50 mg/L ampicillin-resistant plate and grown, cultured at 37° C. for 12 h, and the monoclone was picked out for PCR verification. The primers for PCR verification of the intermediate vector are SEQ ID NO.: 6 and SEQ ID NO.: 7, wherein the forward primer (universal primer T3) is 5′-ATTAACCCTCACTAAAGGGA-3′, the reverse primer (reverse primer of the target) is 5′-AAAC CCCGGGACGTCGGCGAAC TT-3′.

The total volume of the PCR reaction was 15 μL, including the colony template, 2×Taq Mix (purchased from Tsingke) 7.5 μL, ddH₂O 5.5 μL, and the forward and reverse primers each 1 μL.

PCR reaction conditions were as follows: (1) pre-denaturation at 94° C., for 5 min, (2) denaturation at 94° C., for 30 s, (3) annealing at 53° C., for 30 s, (4) extension at 72° C., for 50 s, (5) 38 cycles, (6) extension at 72° C., for 5 min, (7) storage at 4° C.

After the reaction, 1% agarose gel electrophoresis was performed to verify the correct band. The colonies were expanded and cultured, and the plasmid was extracted and sent to Hangzhou Qingke Biotechnology Company for sequencing. Sequencing results showed that the OsCKX11 target had been successfully connected to the intermediate vector SK-gRNA.

The gene sequence of SEQ ID NO.: 6 and SEQ ID NO.: 7 is:

SEQ ID NO.: 6, forward primer: ATTAACCCTCACTAAAGGGA;
SEQ ID NO.: 7, reverse primer: AAAC CCCGGGACGTCGGCGAACT T.

Ligation of OsCKX11 target to final vector pC1300-Cas9.

Recombinant intermediate vector was double digested by using KpnI and Bgl II (purchased from Takara company, specific usage and dosage may be referred to product instruction manual), and the final vector pC1300-Cas9 was double digested by using KpnI and BamHI (purchased from Takara company, specific usage and dosage may be referred to product instruction manual), Bgl II and BamHI were a pair of isocaudomers. The digested product was subjected to electrophoresis by using 1% agarose gel, a band of about 500 bp was recovered from the recombinant intermediate vector, and a band of about 14600 bp was recovered from the final vector pC1300-Cas9.

The recovered target fragments were mixed and ligated to the final vector, ligated to the T4 DNA ligase and transformed into Escherichia coli DH5α, spread on a 50 mg/L kanamycin-resistant plate to grow, cultivated at 37° C. for 12 h, and a single clone was picked for PCR verification. The primer sequences for PCR verification of final vector are SEQ ID NO.: 8 and 9, wherein the forward primer (universal primer T7) is SEQ ID NO.: 8: 5′-ACACTTTATGCTTCCGGCTC-3′, and the reverse primer (target forward primer) is SEQ ID NO.: 9: 5′-AAA C CCCGGGACGTCGGCGAACTT-3′. The system and conditions for PCR verification are the same as above.

The verified and correct colonies were expanded and cultured, and the plasmid was extracted and sent to Hangzhou Qingke Biotechnology Company for sequencing. As shown in FIG. 2, the size of lanes 2, 5, 7, 10, and 11 were correct, which were about 500 bp, and consistent with the positive control. Sequencing results showed that the OsCKX11 target had been successfully ligated to the final vector pC1300-Cas9.

The gene sequences of SEQ ID NO.: 8 and SEQ ID NO.: 8 are:

SEQ ID NO.: 8:
ACACTTTATGCTTCCGGCTC
SEQ ID NO.: 9:
AAAC CCCGGGACGTCGGCGAACTT

Specific Knockout of Gene OsCKX11 of Rice

The verified and correct OsCKX11 target and pC1300-Cas9 vector were sent to Jiangsu Baige Gene Technology Co., Ltd. Plasmid transformation of Agrobacterium tumefaciens, Agrobacterium-mediated transformation of Nipponbare callus, and transgenic rice callus culture were all completed by the company.

### Example 2

Identification of Homozygous Mutants of Osckx11

Extraction of DNA from Transgenic Rice Seedlings

24 transgenic T1 seedlings were obtained in a transgenic cycle of about three months, and DNA was extracted from rice leaves after hardening the seedlings. The kit as used was a plant genomic DNA extraction kit (Shanghai Shenggong Bioengineering Co., Ltd., specific usage and dosage may be referred to the product instructions).

Amplification of Fragments Near the OsCKX11 Gene Target

The OsCKX11 DNA fragments near the target site were amplified by PCR technology. The primers for PCR amplification have following SEQ ID NO.: 10 and SEQ ID NO.: 11.

SEQ ID NO.: 10, forward primer for identification:
5′-ATGGCTGTTTTGGAGGTCCG-3′
SEQ ID NO.: 10, reverse primer for identification:
5′-AGCAGACATGGCACTCGCCG-3′

The total volume of the PCR reaction was 50 μL, including 5 μL of template DNA, 25 μL of 2×KOD Buffer, 7 μL of dNTP, 2 μL of ddH₂O, 5 μL of forward and reverse primers, and 1 μL of KOD FX enzyme. All KOD Buffer, dNTP and KOD FX as used were purchased from TOYOBO Company.

The conditions for PCR reaction were as follows: (1) pre-denaturation at 94° C. for 5 min, (2) denaturation at 98° C. for 10 s, (3) annealing at 62° C. for 30 s, (4) extension at 68° C. for 70 s, (5) 34 cycles, (6) extension at 68° C. for 5 min, (7) storage at 4° C.

The unpurified PCR product was sent to Hangzhou Qingke Biotechnology Company for sequencing.

Analysis of Sequencing Results

Log in to the NCBI (https://www.ncbi.nlm.nih.gov) website, and compare the sequencing results with the deoxynucleotide sequence of gene OsCKX11 as shown in SEQ ID NO.: 1 in the sequence listing. The sequencing results showed that three independent osckx11 homozygous mutant lines were successfully obtained, as shown in FIG. 1B.

The gene sequences SEQ ID NO.: 10 and SEQ ID NO.: 11 are:

SEQ ID NO.: 10:
ATGGCTGTTTTGGAGGTCCG,
SEQ ID NO.: 11:
AGCAGACATGGCACTCGCCG.

### Example 3

Quantification of Cytokinin Content in Osckx11 Homozygous Mutant

Extraction of Cytokinin

T1 generation osckx11 homozygous mutants were harvested and planted in the field. Field sampling was performed on the T2 generation mutants and wild-type flag leaves at the young leaf stage, including three independent mutant lines and 3 biological repeats for each independent line. The sample was ground in liquid nitrogen, about 100 mg of the ground sample was weighed and placed in a 2-mL centrifuge tube (Eppendorf), and the accurate mass was recorded. 1 mL of 80% methanol and a corresponding internal standard ([²H5]tZ, [²H5]tZR, [¹⁵N4]cZ, [¹⁵N4]cZR, [²H6]iP, [²H6]iPR, 45 pg each) were added rapidly. The resulting mixture was vortexed for 2 h at 4° C. Centrifugation was conducted at 4° C. for 10 min at 13000 g. The supernatant was pipetted and transferred to a new 2-mL centrifuge tube, and blown to dry with nitrogen. To the remaining precipitate was again added 1 mL of 80% methanol solution and mixed well at 4° C. The supernatant was pipetted again into the 2 mL centrifuge tube which was dried in the previous step, and blown dry with nitrogen. 300 μL of 30% methanol solution was added and vortexed at 4° C. The well-mixed solution was centrifuged at 4° C. for 10 min at 13000 g. The supernatant was pipetted and the solution was filtered using a 0.22 μm water phase filter membrane. The filtered solution was the hormone extract to be tested.

Quantification of Cytokinin Content

The LC/MS system was used to quantify the content of cytokinin in the test fluid. The extract solution was separated by an ultra-high performance liquid chromatograph (AB SCIEX). The column was equilibrated at 40° C., and 30 μL was loaded for subsequent analysis. The mobile phase for cytokinin detection was prepared as follows: mobile phase A was ultrapure water, mobile phase B was chromatographic grade methanol. Cytokinin detection was conducted by methanol gradient elution. Specifically, 5% ultrapure water for 0-2.5 min; 5-20% chromatographic grade methanol for 2.5-3 min; 20-50% chromatographic grade methanol for 3-12.5 min; 50-100% chromatographic grade methanol for 12.5-13 min; 100% chromatographic grade methanol for 13-15 min; 100-5% chromatographic grade methanol for 15-15.2 min; 5% chromatographic grade methanol for 15.2-18 min. The mobile phase flow rate was 0.3 mL/min.

Cytokinin quantification was performed on QTRAP 5500 mass spectrometry system (AB SCIEX Company) in multi-reaction detection scanning mode. According to the existing literature, the optimized mass spectrometry detection conditions for cytokinin were as follows. Sample atomization pressure was 60 psi; heating pressure was 60 psi; air curtain pressure was 40 psi; positive ion spray voltage was 5000 V; negative ion spray voltage was-4500 V; and the turbine heating temperature was 600° C.

Analysis of Cytokinin Content in Osckx11 Mutant

The results of cytokinin determination were analyzed by AB SCOEX Analyst 1.6.3 software and the original data were obtained. The original data was imported into the AB SCIEX MultiQuant 3.0.2 software for further analysis and processing, and the final data was quantified with reference to the accurate quality of the sample and the internal standard. The results of cytokinin content determination showed that the contents of various cytokinins in the three independent osckx11 mutants increased, of which cZ, tZ, and iP showed a significant increase. Obviously, the loss of function in OsCKX11 gene can up-regulate the cytokinin content in the mutant, and this mutant was confirmed to be a mutant with loss of function of OsCKX11. The results for determination of cytokinin content in the homozygous mutant osckx11 are shown in FIG. 3.

### Example 4

Field Cultivation and Statistics of Osckx11 Homozygous Mutants

Three independent osckx11 homozygous lines T2 generation and Nipponbare wild-type seeds were immersed and germinated, and then planted in the seedling field. After 20 days, the seedlings were transferred to a rice field in Jinhua City, Zhejiang Province. A total of 112 plants were planted in a 1.5 in ×4 in square area in an array of 8×14. Rice seedlings were managed in accordance with general rice planting methods in protected facilities. After 130 days of growth, 20 rice seedlings were randomly selected from each square area (excluding the edge of the square area) to harvest seeds, and the seeds dried at 37° C. for 1 week, and agronomic characteristics were counted.

About 20 plants were chosen from three independent mutant lines, with 4 panicles for each plant. The grain number per panicle and the number of primary branches of a total of 80 lines were counted. After that, the shriveled seeds were removed and threshed, with each plant as a unit, weighed, and the SC-G automatic seed test analyzer (Wanshen Company) was used to count the number of seeds per plant. The wild-type plants were counted in the same way.

### Example 5

Analysis of Panicle Traits of Osckx11 Homozygous Mutants

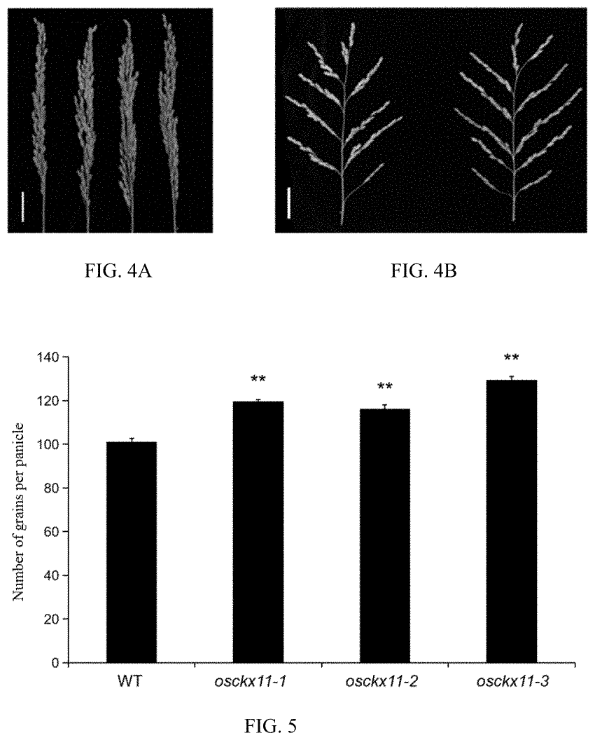

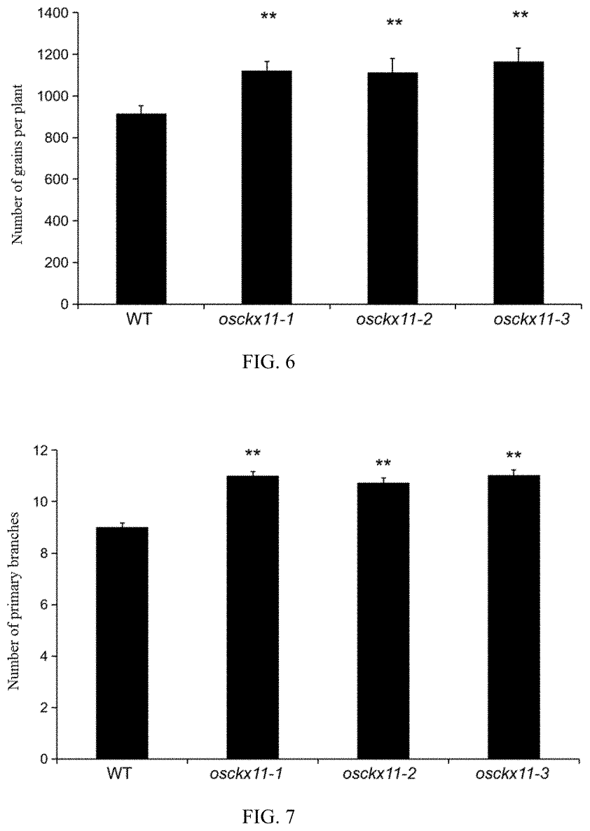

The results showed that the mature panicles of the osckx11 mutant were larger than the wild type, as shown in FIG. 4. The left panicle is the Nipponbare wild type, and the right is three independent osckx11 mutant lines. FIG. 5 is a statistical diagram of the grain number per panicle of the homozygous mutant of osckx11 provided by the embodiment of the present disclosure. WT is the Nipponbare wild type, and osckx11-1, osckx11-2 and osckx11-3 are three independent osckx11 mutant lines. The results show that the wild type has about 101 grains per single panicle, while osckx11 mutant has 115 to 130 grains on a single panicle, showing a significant increase by about 15%-30% when compared with wild-type. Further statistics was conducted on the grain number per plant. FIG. 6 is a statistical diagram of the grain number per plant for the osckx11 homozygous mutant provided by the embodiment of the present disclosure. The grain number per plant for the wild type is 924 and 945 per plant, while the osckx11 mutant has 1114-1166 grains per plant, representing a significant increase of about 20.6%-26.2% compared with WT.

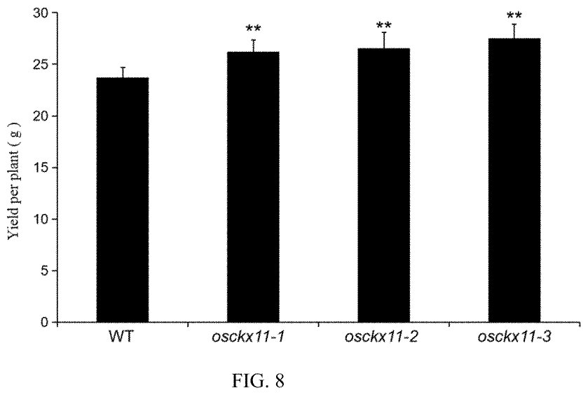

The statistical results for the number of primary branches show that the number of primary branches on the wild-type is 9, while the number of primary branches on the osckx11 mutant is 10.7-11, representing a significant increase of 18.9%-22.2% compared to WT, as shown in FIG. 7. Therefore, the increase in the grain number per panicle is caused by the increase in the number of primary branches, and the increase in the grain number per panicle leads to an increase in the yield of the osckx11 mutant per plant, which increases by about 10%-16%, as shown in FIG. 8.

The above descriptions are merely preferred embodiments of the present disclosure and are not intended to limit the present disclosure. Any modification, equivalent replacement and improvement made within the spirit and principle of the present disclosure shall fall within the protection of the present disclosure.

1-9. (canceled)

10. A plant genetic transformation vector constructed by using a gene for controlling rice grain number per panicle, wherein the plant genetic transformation vector comprises an expression vector for up-regulating expression, and the expression vector for up-regulating expression comprises a recombinant promoter or an expression vector for construction and fusion of organ-specific promoter; or
the plant genetic transformation vector further comprising a DNA sequence consisting of the sequence of SEQ ID NO: 1, or a DNA sequence having 90% or more homology with the sequence of SEQ ID NO: 1, or an allele or a gene derivative with one or more bases being altered produced by base substitution, deletion, or addition, or a DNA molecule capable of hybridizing with the DNA sequence of SEQ ID NO: 1.

11. The plant genetic transformation vector according to claim 10, wherein the gene for controlling rice grain number per panicle has the nucleotide sequence of SEQ ID NO: 1.

12. The plant genetic transformation vector according to claim 10, wherein the encoded protein has the nucleotide sequence of SEQ ID NO: 2.

13. The plant genetic transformation vector according to claim 12, wherein the amino acid sequence of the encoded protein is SEQ ID NO: 3; or
the encoded protein further comprises an amino acid sequence having a than 90% or more of homology with the amino acid sequence of SEQ ID NO: 3; or
the encoded protein further comprises proteins and protein analogs with one or more amino acid being altered, produced by amino acid substitution, deletion, and addition based on the amino acid sequence of SEQ ID NO: 3; or
the encoded protein further comprises a fusion protein formed by ligating the protein of SEQ ID NO: 3 to other tag proteins.

14. The plant genetic transformation vector according to claim 10, wherein the plant genetic transformation vector further comprises an expression vector for down-regulating expression of the gene of SEQ ID NO: 3 by using CRISPR/Cas9 technology, T-DNA insertion technology, EMS mutagenesis, RNA interference technology, gene silencing technology;
and the plant genetic transformation vector up-regulates or down-regulates the expression level or activity of the protein of SEQ ID NO: 3 through a relevant protein regulator.

15. A recombinant bacteria, plant callus and cell line expressed by the plant genetic transformation vector according to claim 10.

16. The recombinant bacteria, plant callus and cell line according to claim 15, wherein the gene for controlling rice grain number per panicle has the nucleotide sequence of SEQ ID NO: 1.

17. The recombinant bacteria, plant callus and cell line according to claim 15, wherein the encoded protein has the nucleotide sequence of SEQ ID NO: 2.

18. The recombinant bacteria, plant callus and cell line according to claim 17, wherein the amino acid sequence of the encoded protein is SEQ ID NO: 3; or
the encoded protein further comprises an amino acid sequence having a than 90% or more of homology with the amino acid sequence of SEQ ID NO: 3; or
the encoded protein further comprises proteins and protein analogs with one or more amino acid being altered, produced by amino acid substitution, deletion, and addition based on the amino acid sequence of SEQ ID NO: 3; or
the encoded protein further comprises a fusion protein formed by ligating the protein of SEQ ID NO: 3 to other tag proteins.

19. The recombinant bacteria, plant callus and cell line according to claim 15, wherein the plant genetic transformation vector further comprises an expression vector for down-regulating expression of the gene of SEQ ID NO: 3 by using CRISPR/Cas9 technology, T-DNA insertion technology, EMS mutagenesis, RNA interference technology, gene silencing technology;
and the plant genetic transformation vector up-regulates or down-regulates the expression level or activity of the protein of SEQ ID NO: 3 through a relevant protein regulator.
